(19) United States
(12) Patent Application Publication
(10) Pub. No.: US 2020/0114048 A1
(43) Pub. Date: Apr. 16, 2020

(54) ABSORBABLE IMPLANT MATERIAL COMPOSED OF MAGNESIUM OR A MAGNESIUM ALLOY CONTAINING DOPED NANODIAMONDS

(71) Applicant: Helmholtz-Zentrum Geesthacht Zentrum für Material-und Küstenforschung GmbH, Geesthacht (DE)

(72) Inventor: Hajo Dieringa, Luneburg (DE)

(73) Assignee: Helmholtz-Zentrum Geesthacht Zentrum für Material-und Küstenforschung GmbH, Geesthacht (DE)

(21) Appl. No.: 16/599,092

(22) Filed: Oct. 10, 2019

(30) Foreign Application Priority Data
Oct. 10, 2018 (EP) 18199678.6

(51) Int. Cl. A61L 31/12; A61L 31/02; A61L 31/14; B01J 19/10

(52) U.S. Cl. CPC A61L 31/124; A61L 31/022; A61L 2430/02; A61L 31/148; B01J 19/10; A61L 31/024

(57) ABSTRACT

An absorbable implant material comprising homogeneously distributed Fe-doped nanodiamonds in a matrix composed of magnesium or a magnesium alloy and a method for the production thereof is provided. The absorbable implant material is produced by a method in which magnesium or a magnesium alloy is melted, Fe-doped nanodiamonds are added to the melt, and the melt composed of magnesium or a magnesium alloy that has been provided with Fe-doped nanodiamonds is subjected to an ultrasound treatment.

### CROSS-REFERENCE TO RELATED APPLICATION

The present application claims priority to European Application No. 18199678.6, filed Oct. 10, 2018, the entire disclosure of which is hereby incorporated by reference in its entirety.

### FIELD OF THE INVENTION

The present invention relates to an implant material composed of magnesium or a magnesium alloy and to a method for the production thereof.

### BACKGROUND OF THE INVENTION

At present, metal implants composed of medical-grade steel or titanium are used both in veterinary medicine and in human medicine to treat fractures of weight-bearing long bones. In terms of their mechanical behaviour, said implants are, however, more rigid than bone, and this can lead to the phenomenon of stress shielding. For these and other reasons, relevant implants are generally removed after they have fulfilled their function, and this can place stress on the patient owing to the required anaesthesia and the renewed tissue trauma.

Absorbable implants are of increasing interest for fracture treatment. The goal is that, as the healing bone increases in strength, the implants undergo a stress adjustment via a slow decrease in their stability. The use of the absorbable implants that are available to date and are composed of different polymers does not work optimally because of their low strengths on the stressed bone. By contrast, magnesium and its alloys exhibit, in comparison with other metallic implant materials, an elastic modulus similar to bones and favourable tensile strength and compressive strength. Magnesium and its alloys have higher strengths and a greater elastic modulus than absorbable polymers and are therefore the focus of scientific research. Bioabsorable implants, especially composed of magnesium or a magnesium alloy, for the treatment of bone fractures are, for example, known from EP 2 318 057 B1 and publications cited therein or from DE 10 2005 060 203 A1.

Absorbable implants are used not just for fracture treatment. Nowadays, implants composed of magnesium and its alloys are used particularly frequently as stents, which serve for the treatment of stenoses (vascular constrictions). Stents have a tubular or hollow-cylindrical base lattice that is open at both longitudinal ends. The tubular base lattice of such an endoprosthesis is inserted into the vessel to be treated and serves to support the vessel. Biodegradable stents composed of magnesium or magnesium alloy are, for example, known from EP 2 198 898 B1 and publications cited therein.

However, a disadvantage of the known implants is that the position of the implant during the medical procedure for the implantation thereof or immediately thereafter could only be ascertained by means of X-ray examinations. Implant absorption, too, can as yet only be tracked by means of X-ray examinations. Said examinations are comparatively complex and cost-intensive.

It is an object of the present invention to provide an implant material composed of magnesium or a magnesium alloy and a method for the production thereof, the position of which during the medical procedure for the implantation thereof and the absorption of which in the body of the patient can be tracked in a simple manner.

### SUMMARY OF THE INVENTION

The present invention relates to an absorbable implant material composed of magnesium or a magnesium alloy and to a method for the production thereof. A disadvantage of known absorbable implants is that the position of the implant during the medical implantation procedure and immediately thereafter can only be tracked by means of X-ray examinations. According to the invention, what is provided is an absorbable implant material comprising homogeneously distributed Fe-doped nanodiamonds in a matrix composed of magnesium or a magnesium alloy. Fe-doped nanodiamonds are harmless to organisms. This allows the detection of the implant material in the blood plasma of the patient by means of magnetic resonance imaging.

According to the invention, the absorbable implant material according to the invention is produced by a method in which magnesium or a magnesium alloy is melted, Fe-doped nanodiamonds are added to the melt, and the melt composed of magnesium or a magnesium alloy that has been provided with Fe-doped nanodiamonds is subjected to an ultrasound treatment.

The object is achieved by an implant material according to claim 1, comprising homogeneously distributed Fe-doped nanodiamonds in a matrix composed of magnesium or a magnesium alloy. The object is also achieved by a method for producing an implant material according to claim 6, in which magnesium or a magnesium alloy is melted, Fe-doped nanodiamonds are added to the melt, and the melt composed of magnesium or a magnesium alloy that has been provided with Fe-doped nanodiamonds is subjected to an ultrasound treatment.

### DETAILED DESCRIPTION OF THE INVENTION

Fe-doped nanodiamonds (Fe-NDs) have, for example, been disclosed as protein labels by B.-R. Lin et al. “Fe Doped Magnetic Nanodiamonds Made by Ion Implantation as Contrast Agent for MRI” Scientific Reports (2018) 8:7058. To date, Fe-doped nanodiamonds have been used in research for the visualization of biological cellular processes. Fe-doped nanodiamonds are harmless to organisms and can, as contrast agent, render biological processes visible.

For the production of Fe-doped nanodiamonds, we refer to “Fe Doped Magnetic Nanodiamonds Made by Ion Implantation as Contrast Agent for MRI”. Nanodiamonds are well-known and can, for example, be purchased from Sigma-Aldrich. Fe ions can be easily implanted into said nanodiamonds. To this end, the nanodiamonds are preferably suspended in demineralized water and the suspension is subsequently applied to a silicon wafer. The Fe ions can then be implanted into the nanodiamonds by sputtering. In this process, preference is given to using an energy of about 100-200 keV, such as about 150 keV, and a dose of about 1×10¹⁵ atoms/cm² to 1×10¹⁶ atoms/cm², such as about 5×10¹⁵ atoms/cm².

The implant material according to the invention that is composed of magnesium or a magnesium alloy and contains homogeneously distributed Fe-doped nanodiamonds can be produced by means of introduction of the Fe-doped nanodiamonds into a melt of the implant material. Afterwards, said implant material can be extruded or be processed by means of powder-metallurgy methods such as MIM technology to form implant bodies. The position thereof in the body of the patient can then be detected with the aid of magnetic resonance imaging (MRI) or by other means.

As the implant material is absorbed in the body of the patient, the Fe-doped nanodiamonds get into the bloodstream. The Fe-doped nanodiamonds are gradually excreted from the body. The degradation of the implant material can likewise be tracked with the aid of magnetic resonance imaging (MRI) or by other means.

If a magnesium alloy is used as matrix material, preference is given to using alloy elements considered to be non-hazardous to health. Preference is given to using magnesium alloys having alloy elements selected from the group consisting of lithium, calcium, potassium, strontium, barium, scandium, yttrium, lanthanum, praseodymium, neodymium, samarium, europium, gadolinium, dysprosium, silicon, copper, zinc, gallium, gold, silver, bismuth, iron and combinations thereof. Greater preference is given to using magnesium alloys as described in DE 10 2016 007 176 A1 or DE 10 2016 119 227 A1 (corresponding to International Publication No. WO 2018/069230), which are hereby incorporated herein by reference in their entirety.

According to the invention, the implant material is produced by melting magnesium or a magnesium alloy, adding nanodiamonds to the melt and subjecting the melt composed of magnesium or a magnesium alloy that has been provided with nanodiamonds to an ultrasound treatment.

Such a method for homogeneously distributing nanoparticles in a melt composed of magnesium or a magnesium alloy is described in the article by H. Dieringa et al. “Ultrasound Assisted Casting of an AM60 Based Metal Matrix Nanocomposite, Its Properties, and Recyclability” in Metals 2017, 7, 338, which is hereby incorporated herein by reference in its entirety.

In a preferred method for producing the implant material according to the invention, magnesium or a magnesium alloy is preferably melted under a protective gas and with stirring in a permanent mould situated in an oven in a first step, the melt is admixed with the Fe-doped nanodiamonds in a second step and the nanodiamonds introduced into the melt are dispersed and deagglomerated by means of a sonotrode in a third step. A similar method is, for example, described in H. Dieringa et al. “Ultrasound Assisted Casting of an AM60 Based Metal Matrix Nanocomposite, Its Properties, and Recyclability” in Metals 2017, 7, 338, to which full reference is made here. The melt is preferably mechanically stirred, preferably at 150 to 250 rpm. Thereafter, the Fe-doped nanodiamonds are added to the melt. After addition of the Fe-doped nanodiamonds, the melt is treated with ultrasound. To this end, preference is given to introducing a sonotrode into the melt. The ultrasound treatment preferably takes place over a period of 1 min to 10 min, more preferably 2 min to 5 min.

It is further preferred that the permanent mould containing the melt is immersed in a water bath after removal of the stirrer and the sonotrode. The melt thus solidifies from “bottom to top”, resulting in the avoidance of shrink-hole formation.

The implant material according to the invention preferably comprises homogeneously distributed Fe-doped nanodiamonds in a matrix composed of magnesium or a magnesium alloy in an amount of 0.01% to 3% by weight, preferably 0.5% to 1.5% by weight, based on the weight of magnesium or magnesium alloy. The nanodiamonds preferably have a particle size of 1 to 20 nm, preferably 3 to 8 nm.

The implant material thus produced can be subsequently further processed in the usual manner. For example, the implant material can be re-melted and then cast into the desired mould to form an implant body. The material can also be extruded in order to manufacture implants from the extrudate. Alternatively, the implant material can be further processed to form powder and further processed by means of metal injection moulding (MIM) to form an implant body.

The implant material according to the invention can also be processed to form a metallic implant body with the aid of MIM technology. With the aid of MIM technology, it is possible to manufacture small, complex and precisely shaped metal components in a near-net-shape process. MIM technology is part of the so-called powder-metallurgy methods, in which the starting material used for the component to be produced is not a solid metal body, but fine metal powder. MIM stands, then, for metal injection moulding. In the MIM method, the metal powder is rendered flowable by addition of thermoplastic binders and the flowable mixture is introduced into an injection mould. After moulding, the binder portion is removed and the component is sintered. Magnesium components can be produced with the aid of MIM technology according to the method described in M. Wolff et. al. “Magnesium powder injection moulding for biomedical application”, Powder Metallurgy, 2014 (Vol. 57, No. 5), 331-340, which is hereby incorporated herein by reference in its entirety.

When using MIM technology, the binder provides for a temporary bond during casting or moulding and ensures the stability of the component until final compaction of the metal powder by sintering. Some of the binder is generally already removed before sintering, for example with the aid of a solvent (solvent debinding). The rest of the binder decomposes under thermal debinding at temperatures of about 300° C. to 500° C. and escapes in gaseous form.

1. An implant material comprising homogeneously distributed Fe-doped nanodiamonds in a matrix composed of magnesium or a magnesium alloy.

2. The implant material of claim 1, wherein the homogeneously distributed Fe-doped nanodiamonds are present in the matrix in an amount of 0.01% to 3% by weight, based on the weight of the magnesium or the magnesium alloy.

3. The implant material of claim 2, wherein the homogeneously distributed Fe-doped nanodiamonds are present in the matrix in an amount of 0.5% to 1.5% by weight, based on the weight of the magnesium or the magnesium alloy.

4. The implant material of claim 1, wherein the Fe-doped nanodiamonds have a particle size of 1 to 20 nm.

5. The implant material of claim 4, wherein the Fe-doped nanodiamonds have a particle size of 3 to 8 nm.

6. A method for producing an implant material according to claim 1, comprising:
melting magnesium or a magnesium alloy;
adding Fe-doped nanodiamonds to the melt; and
subjecting the melt composed of magnesium or a magnesium alloy that has been provided with nanodiamonds to an ultrasound treatment, thereby producing the implant material.

7. The method of claim 6, wherein the magnesium or the magnesium alloy is melted under a protective gas and with stirring in a permanent mould situated in an oven in a first step, the melt is mechanically stirred and the Fe-doped nanodiamonds are subsequently added to the melt, and the melt is treated with ultrasound after addition of the Fe-doped nanodiamonds.

8. The method of claim 7, wherein the ultrasound treatment is effected by means of a sonotrode introduced into the melt.

9. The method of claim 6, wherein the ultrasound treatment takes place over a period of 1 minute to 10 minutes.

10. The method of claim 9, wherein the ultrasound treatment takes place over a period of 2 minutes to 5 minutes.

11. The method of claim 7, further comprising transferring the permanent mould is transferred to a water bath, where the melt solidifies, after the ultrasound treatment.

12. The method of claim 6, further comprising re-melting and then casting the implant material into a desired mould in order to yield a metallic implant.

13. The method of claim 6, further comprising extruding the implant material, the extrudate serving as a base material for the manufacture of an implant.

14. The method of claim 6, further comprising converting the implant material into a metallic implant by metal injection moulding technology.
